(19) United States
(12) Patent Application Publication
(10) Pub. No.: US 2020/0205688 A1
(43) Pub. Date: Jul. 2, 2020

(54) MEDICAL TOILET WITH ELECTROCARDIOGRAM

(71) Applicants: David R. Hall, Provo, UT (US); Vivek Garg, Sandy, UT (US)

(72) Inventors: David R. Hall, Provo, UT (US); Vivek Garg, Sandy, UT (US)

(21) Appl. No.: 16/233,088

(22) Filed: Dec. 27, 2018

(51) Int. Cl. A61B 5/0408; A61B 5/00; A61B 5/024

(52) U.S. Cl. CPC A61B 5/0408; A61B 5/0006; A61B 5/02444; E03D 11/00; A61B 5/6831; A61B 2562/16; A61B 5/6887

(57) ABSTRACT

The medical toilet includes a first, second, and third electrocardiogram (EKG) lead for collecting EKG measurements from a user while seated on the medical toilet. The toilet may include arm rests, each an EKG leads placed on or within it. The arm rests may include hand grips, each hand grip including one of the EKG leads. The arm rests may have finger slips which include EKG leads. The arm rests may each have a wrist strap which includes one of the EKG leads. The first and second EKG leads may be on a toilet lid. The third EKG lead may be placed on the base of the toilet where a user may place an ankle or it may be on a toilet seat. Alternatively, the third EKG lead may be on a foot pad or within an ankle wrap. A chest strap may include additional EKG leads.

### BACKGROUND

### Field of the Invention

This disclosure relates to toilets and medical devices which collect measurements related to cardiovascular health.

### Background of the Invention

Toilets are used daily to deposit bodily waste. Recently, medical toilets have emerged which collect measurements which are relevant to a user's health status. By collecting these measurements while a user is seated on the toilet, the measurements are collected on a regular basis and the user is not significantly inconvenienced. Typically, medical toilets may collect a variety of health-related measurements, for example, body weight, body temperature, and analyze bodily waste. However, a user must seek medical attention in a clinical setting to collect measurements which are directly indicative of more life-threatening diseases, such as cardiovascular disease. A medical toilet which collects electrocardiogram (hereinafter “EKG”) measurements would be useful and may save lives.

### BRIEF SUMMARY OF THE INVENTION

We disclose a medical toilet which includes at least three EKG leads. Two of the EKG leads may be positioned on the medical toilet to contact a user's upper body when the user is seated on the medical toilet while the third EKG lead may be positioned on the medical toilet to contact a user's left lower extremity. Three of the at least three leads may function as limb leads I, II, and III as known in the art and which form the points known as Einthoven's Triangle.

In one embodiment, the medical toilet includes arm rests. Each arm rest includes one of the at least three EKG leads. In another embodiment, the arm rests include hand grips with one of the at least three EKG leads included in each of the hand grips. In another embodiment, the arm rests include finger slips with one of the at least three EKG leads included in each finger slip. A pulse oximeter may also be included in a finger slip. In another embodiment, two of the at least three EKG leads may be positioned on a toilet lid such that the user may lean back against the toilet lid while seated on the toilet and thereby come in contact with the two EKG leads.

The third EKG lead may be positioned on a foot pad. A marker may be placed on the foot pad to instruct a user where to position the user's left foot to contact the third EKG lead. In another embodiment, the third EKG lead may be disposed on the base of the toilet such that a user may place a left ankle against the toilet base to contact the EKG lead. In another embodiment, the third EKG lead may be disposed on the left side of the toilet seat such that a user's left thigh contacts the third EKG lead when the user is seated on the medical toilet.

In another embodiment, the medical toilet includes two arm rests with a wrist band on each of the arm rests. The first and second of the at least three EKG leads may be disposed on each of the wrist bands. An ankle band may be disposed on the left side of the toilet base with the third electrode disposed therein. A user may sit on the medical toilet and wrap a wrist band around each wrist and the ankle band around a left ankle to collect an EKG measurement.

In some embodiments, the medical toilet may include a chest strap which includes six precordial leads V1, V2, V3, V4, V5 and V6. The chest strap may comprise an elastic material which may adjust to the size of the user in order to properly position the six precordial leads on the user's chest.

The medical toilet may include a controller. The controller may include non-transitory computer readable medium which stores instructions for analyzing the signals collected by the EKG leads. The controller may also store the signals for later use. The instructions may include steps required to identify abnormal cardiac rhythms and determine heart rate. The instructions may also include steps required to analyze a signal from a pulse oximeter to calculate a percent oxygen saturation of the user's blood. In some embodiments, three of the at least three leads may function as augmented leads aV_{L}, aV_{R}, and aV_{F} as well as function as limb leads I, II, and III. The instructions may include steps required to interpret the signal from the three leads as either limb leads or augmented leads and to combine the signal from the three leads with the signals from the precordial leads to result in a 12 lead EKG reading.

### BRIEF DESCRIPTION OF THE DRAWINGS

In order that the advantages of the invention will be readily understood, a more particular description of the invention briefly described above will be rendered by reference to specific embodiments illustrated in the appended drawings. Understanding that these drawings depict only typical embodiments of the invention and are not therefore to be considered limiting of its scope, the invention will be described and explained with additional specificity and detail through use of the accompanying drawings.

FIG. 1 illustrates a perspective view of a medical toilet with EKG leads on arm rests and on a foot pad according to an embodiment of the disclosure.

FIG. 2A illustrates a perspective view of a medical toilet with EKG leads on arm rests and on the toilet base according to an embodiment of the disclosure.

FIG. 2B illustrates a close-up view of the EKG lead on the toilet base presented in FIG. 2A.

FIG. 3 illustrates a perspective view of a user seated on a medical toilet which includes EKG leads on a chest strap according to an embodiment of the disclosure.

FIG. 4 illustrates a perspective view of a medical toilet with EKG leads on hand grips and on a foot pad according to an embodiment of the disclosure.

FIG. 5A illustrates a perspective view of a medical toilet with EKG leads in finger slips and on a foot pad according to an embodiment of the disclosure.

FIG. 5B illustrates a close up view of a finger slip on the toilet of FIG. 5A.

FIG. 6A illustrates a perspective view of a medical toilet with EKG leads on wrist bands and on an ankle band according to an embodiment of the disclosure.

FIG. 6B illustrates a close-up view of a user's arm with the wrist band shown in FIG. 6A engaged on the user's wrist.

FIG. 7 illustrates a perspective view of a user seated on the medical toilet of FIG. 6A.

FIG. 8 illustrates a perspective view of a medical toilet with EKG leads on arm rests and on a toilet seat according to an embodiment of the disclosure.

FIG. 9 illustrates a perspective view of a medical toilet with EKG leads on the toilet lid and toilet seat.

### DETAILED DESCRIPTION OF THE INVENTION

Definitions:

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “user” means the individual who comes in contact with the toilet disclosed herein and/or who deposits bodily waste into the toilet disclosed herein.

As used herein, “left” means the side of the toilet which is on a user's left side when a user is seated on the medical toilet. With respect to the drawings, left is the viewer's right side.

As used herein, “right” means the side of the toilet which is on a user's right when a user is seated on the medical toilet. With respect to the drawings, right is the viewer's left side.

While this invention is susceptible of embodiment in many different forms, there are shown in the drawings, which will herein be described in detail, several specific embodiments with the understanding that the present disclosure is to be considered as an exemplification of the principals of the invention and is not intended to limit the invention to the illustrated embodiments.

We disclose a medical toilet which may include electrocardiogram (hereinafter “EKG”) leads which may be used to collect measurements which are relevant to a user's cardiovascular health. The medical toilet may resemble a traditional water toilet and include a base and a toilet seat. The medical toilet may include two arm rests on which a user may rest a left and a right arm during use. The medical toilet may include a controller which includes a non-transitory computer readable medium.

The medical toilet may include at least three EKG leads which may function as limb leads I, II, and III as known in the art and which form the points known as Einthoven's Triangle. Two of the EKG leads may be positioned to contact a user's upper body, for example, the user's upper extremities. These leads may function as limb leads I and II. A third EKG lead may be positioned to be in contact with a user's lower body, for example, a lower extremity. The third lead may function as limb lead III. In an example, the third EKG lead may be disposed on the left lower quadrant of the medical toilet.

Each of the EKG leads may be in electrical communication with the controller. The non-transitory computer readable medium may include instructions for interpreting a signal collected by the EKG leads. In addition, the non-transitory computer readable medium may include instructions for storing the signal collected by the EKG leads on the computer's memory.

In some embodiments, the non-transitory computer readable medium may include instructions for analyzing the signals collected by the EKG leads to identify an irregular cardiac rhythm. In some embodiments, the non-transitory computer readable medium may include instructions for determining a user's heart rate.

In some embodiments, the first and the second EKG leads are each disposed on one of two arm rests included on the medical toilet. A user may sit on the medical toilet and place a left and a right arm flat against the left and the right arm rest respectively. The user's arms or hands may come in contact with the first and the second EKG leads within the arm rests.

In another embodiment, medical toilet may include two arm rests which include hand grips at the distal (far) end of each of the arm rests. A user may place his or her arms on the arm rests and grasp a hand grip with each hand. The first and second EKG leads are each disposed within one of the hand grips.

In another embodiment, the medical toilet may include a toilet lid. The first and the second electrode may be disposed on the toilet lid. When a user is seated on the medical toilet, the user may lean backwards against the toilet lid placing his or her back against the first and the second EKG leads disposed on or within the toilet lid.

In some embodiments, the medical toilet includes a first and a second finger slip. The first and the second finger slips may be disposed on the ends of two arm rests on the medical toilet. The first EKG lead may be within a first finger slip and the second EKG lead may be within the second finger slip. When a user is seated on the medical toilet, the user may place his or her arms on the arm rests and insert a finger on each hand into one of the two finger slips. The first and the second EKG leads may come in contact with the user's fingers and, with the third electrode, collect EKG measurements.

Additionally, one of the finger slips may also include a pulse oximeter. The pulse oximeter may be in electrical communication with the controller. The pulse oximeter may measure the percent oxygen saturation in the user's blood when the user inserts a finger into the finger slip which includes the pulse oximeter. The signal may be transmitted to the controller for storage and analysis using instructions stored on the non-transitory computer readable medium.

The third EKG lead may be disposed in a variety of locations on the medical toilet such that a user's lower extremity may come in contact with the third EKG lead when the user is seated on the medical toilet. In one embodiment, the third EKG lead is disposed on a left side of the toilet base. A user may position a lower leg, for example, an ankle, against the left side of the toilet base in contact with EKG lead. The signal measured by the third EKG lead may be transmitted to the controller for storage and analysis. With the lower extremity in contact with the third EKG lead along with the signals acquired by the first and the second EKG lead, the instructions stored on the non-transitory computer readable medium may provide an analysis of the signals that is relevant to the user's cardiovascular health.

In another embodiment, the medical toilet includes a foot pad. In some embodiments, the foot pad may be the pressure-sensing pad disclosed in U.S. patent application Ser. No. 16/173,171 filed on Oct. 29, 2018 which is herein incorporated by reference in its entirety. The foot pad may be positioned in front of the base of the medical toilet such that a user seated on the medical toilet may place both feet on the foot pad. In some embodiments, the foot pad includes markers to inform the user where to place each foot. The foot pad may include the third EKG lead. In the embodiment which include markers, the third EKG lead may be positioned within a marker so that the user's left foot will come in contact with the third EKG lead when the user places the foot on the marker. With the user's left foot in contact with the third EKG lead along with the signals acquired by the first and the second EKG lead, the instructions stored on the non-transitory computer readable medium may provide an analysis of the signals that is relevant to the user's cardiovascular health.

In another embodiment, the medical toilet includes two arm rests, each with a wrist band attached. The first and the second EKG leads may be disposed within the two wrist bands. An ankle band may be positioned on the base of the medical toilet and the third EKG lead may be disposed within the ankle band. A user may sit on the medical toilet and place his or her arms on the arm rests. The user may wrap each of the wrist bands around a wrist and the ankle band around an ankle. The first, second, and third EKG leads may collect measurements and transmit the signal to the controller. The instructions stored on the non-transitory computer readable medium may provide an analysis of the signals that is relevant to the user's cardiovascular health.

In another embodiment, the medical toilet includes precordial EKG leads V1, V2, V3, V4, V5 and V6 in addition to the first, the second, and the third EKG leads. The precordial EKG leads may be disposed on a chest strap. The chest strap may be constructed of an elastic fabric. The user may be seated on the medical toilet and wrap the chest strap around his or her chest securing the end of the chest strap to the medical toilet. The chest strap may be adjustable so that the user may position the precordial EKG leads in their proper positions on the user's chest as is known in the art. The precordial EKG leads may collect measurements and transmit the signal to the controller along with the signals collected by the first, second, and third EKG leads. The instructions stored on the non-transitory computer readable medium may provide an analysis of the signals that is relevant to the user's cardiovascular health.

In some embodiments which include the precordial EKG leads, the instructions stored on the non-transitory computer readable medium may comprise steps for alternatively designating the first, second, and third electrocardiogram leads as either augmented leads aV_{L}, aV_{R}, and aV_{F} or as limb leads I, lead II, and lead III respectively. The instructions may combine the signals from the first, second, and third leads when defined as limb leads with the interpretation of the signals when the first second, and third leads are defined as augmented leads. The signals from the precordial EKG leads may further be included in the calculations to acquire a 12 lead EKG reading which may provide a more complete assessment of the user's cardiac function.

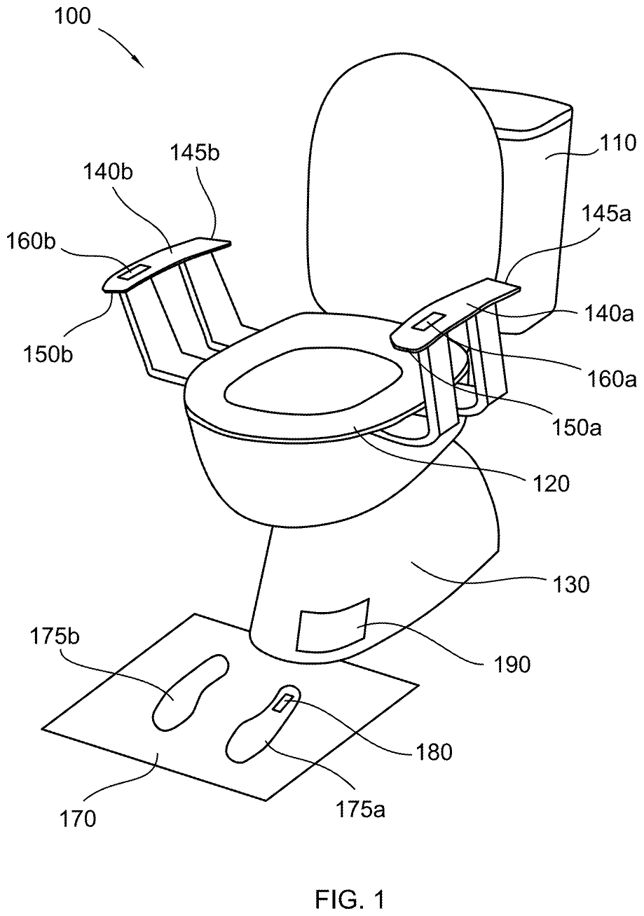

Referring now to the drawings, FIG. 1 illustrates medical toilet 100, which, like traditional toilets, includes tank 110, toilet seat, 120, and toilet base 130. The toilet also includes arm rest 140a and arm rest 140b. Arm rest 140a includes proximal end 145a and distal end 150a while arm rest 140b includes proximal end 145b and distal end 150b. Distal end 150a of arm rest 140a includes EKG lead 160a while distal end 150b of arm rest 140b includes EKG lead 160b. A user seated on toilet 100 may place a wrist on each of EKG leads 160a and 160b when collecting an EKG measurement. Foot pad 170 is included in medical toilet 100 and includes left foot marker 175a and right foot marker 175b. EKG lead 180 is disposed upon left foot marker 175a. A user may place a left foot on left foot marker 175a to bring the user's foot in contact with EKG lead 180 when collecting EKG measurements. EKG leads 160a, 160b and 180 are in electrical communication with controller 190 which, in this embodiment, is disposed within toilet base 130. EKG leads 160a, 160b and 180 collect measurements and transmit the measurements to controller 190 for analysis and storage.

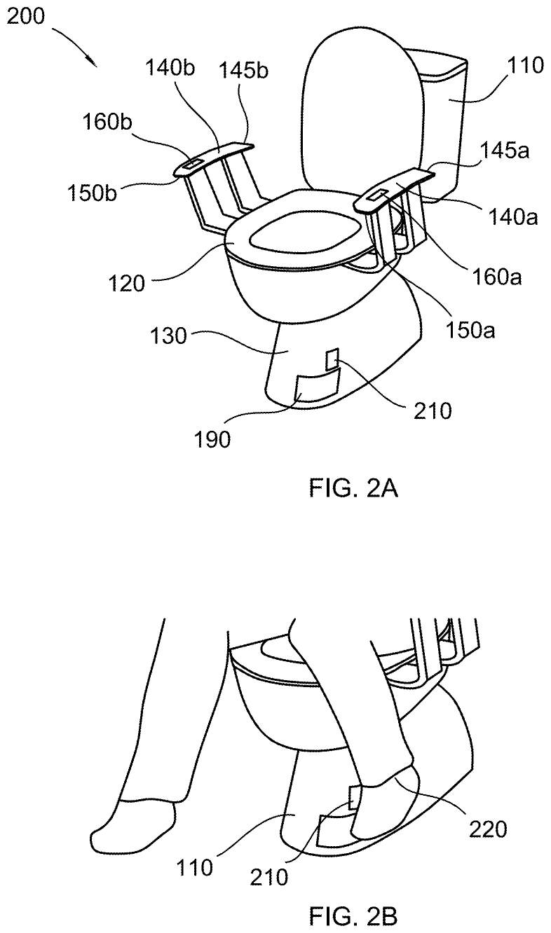

FIG. 2A illustrates medical toilet 200, another embodiment of the disclosure. Like medical toilet 100, medical toilet 200 includes EKG leads 160a and 160b disposed on distal ends 150a and 150b of arm rests 140a and 140b respectively. In contrast with medical toilet 100, medical toilet 200 includes EKG lead 210 on a lower left quadrant of toilet base 130. A user seated on medical toilet 200 may place a wrist on each of EKG leads 160a and 160b when collecting an EKG measurement. In addition, FIG. 2B illustrates that the user may place the user's left ankle 220 on EKG lead 210 while collecting an EKG measurement. EKG leads 160a, 160b and 210 collect measurements and transmit the measurements to controller 190 for analysis and storage.

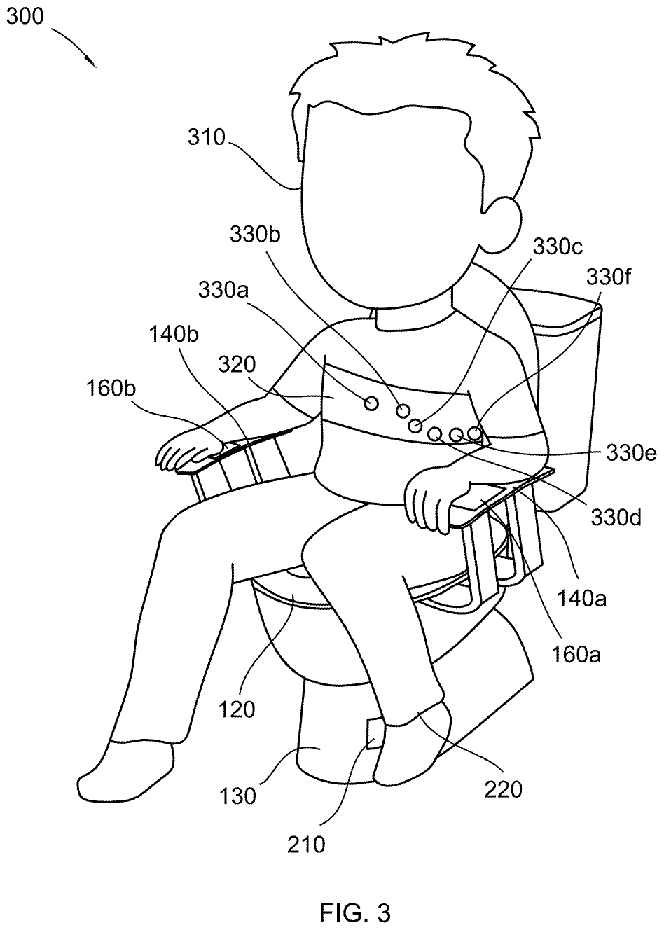

FIG. 3 shows user 310 seated on yet another embodiment of the disclosure, medical toilet 300. Medical toilet 300 resembles medical toilet 200 first presented in FIG. 2A. Additionally, medical toilet 300 includes chest strap 320 which includes six additional EKG lead. EKG leads 330a, 330b, 330c, 330d, 330e, and 330f are positioned on chest strap 320 according to proper placement of precordial leads V1, V2, V3, V4, V5, and V6 respectively. Chest strap 320 is adjustable in length and is elastic to properly fit users of various chest sizes. The user has stretched chest strap 320 across his chest and secured the end of chest strap 320 such that the length of chest strap 320 places EKG leads 330a-f in their appropriate positions. EKG leads 160a, 160b and 310a-f collect measurements and transmit the measurements to controller 190 for analysis and storage.

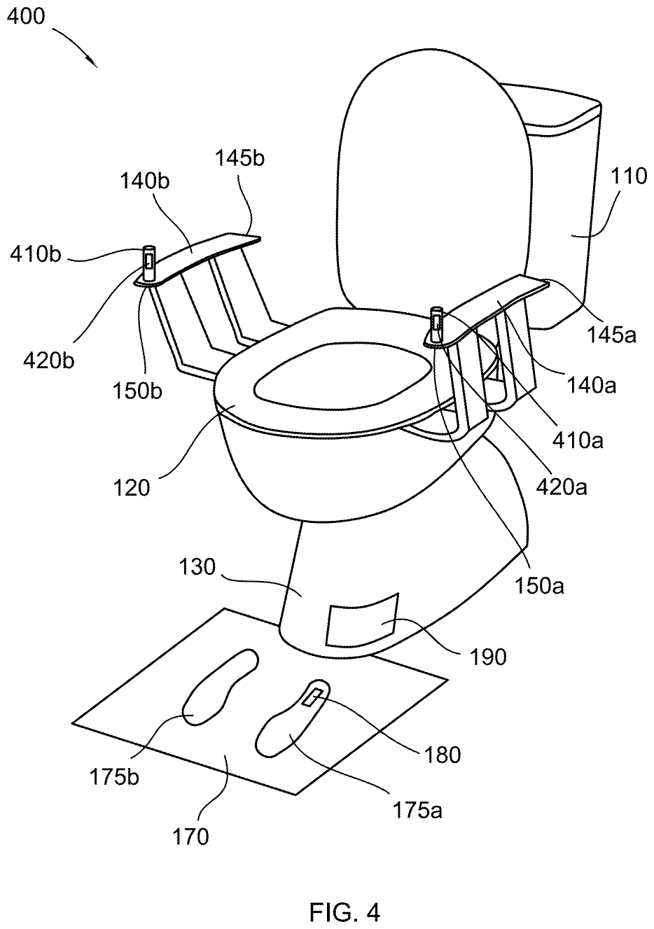

FIG. 4 illustrates medical toilet 400 which includes arm rests 140a and 140b similar to those of medical toilet 100 presented in FIG. 1. However, distal ends 150a and 150b of arm rests 140a and 140b respectively include hand grips 410a and 410b respectively. Also unlike medical toilet 100, EKG leads are not disposed directly on arm rests 140a and 140b. Instead, EKG leads 420a and 420b are disposed on hand grips 410a and 410b respectively. A user seated on toilet 400 may grasp each of hand grips 410a and 410b while taking an EKG reading. In addition, a user may place a left foot on left foot marker 175a to bring the user's left foot in contact with EKG lead 180 while collecting EKG measurements. EKG leads 420a, 420b and 180 are in electrical communication with controller 190 which, in this embodiment, is disposed within toilet base 130. EKG leads 420a, 420b and 180 collect measurements and transmit the measurements to controller 190 for analysis and storage.

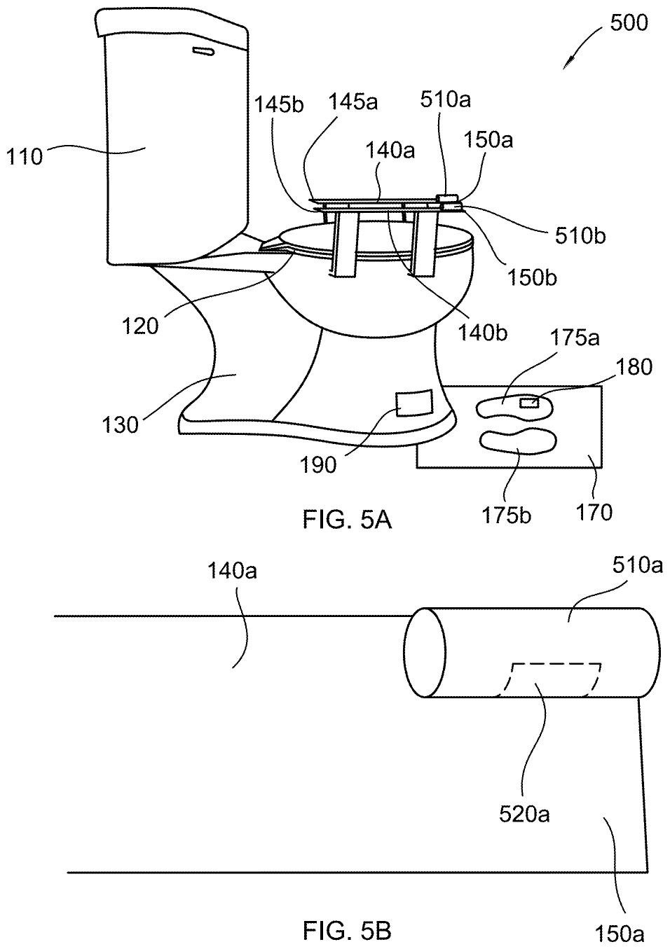

FIG. 5A illustrates medical toilet 500 which resembles medical toilet 100 of FIG. 1. Similar to medical toilet 100, medical toilet 500 includes arm rests 140a and 140b, foot pad 170 which includes left and right foot markers 175a and 175b and EKG lead 180 on left foot marker 175a. However, unlike medical toilet 100, EKG leads are not disposed directly on arm rests 140a and 140b. Instead, finger slips 510a and 510b each house an EKG lead. Finger slip 510a is disposed on distal end 150a of arm rest 140a and finger slip 510b is disposed on distal end 150b of arm rest 140a. A user may slide a finger into each of fingers slips 510a and 510b. Each finger may come in contact with one of the EKG leads. In addition, a user may place a left foot on left foot marker 175a to bring the user's foot in contact with EKG lead 180 while collecting EKG measurements. All three EKG leads on medical toilet 500 collect measurements and transmit the measurements to controller 190 for analysis and storage.

FIG. 5B illustrates distal end 150a of arm rest 140a on medical toilet 500. A close-up view of finger slip 510a is shown which illustrates EKG lead 520a within finger slip 510a. A second EKG electrode is likewise positioned within finger slip 510b on arm rest 140b.

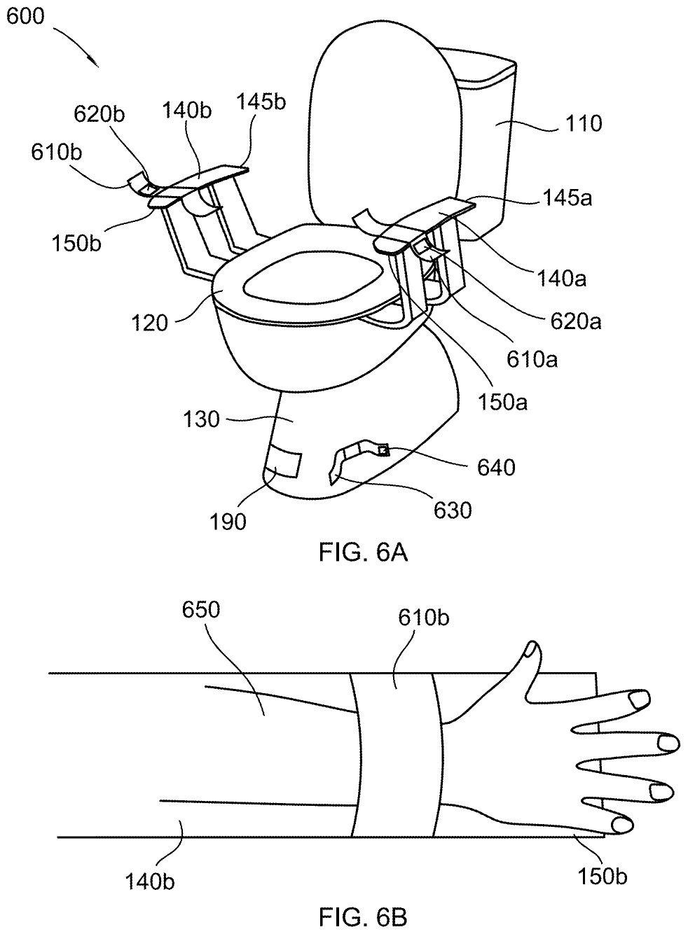

FIG. 6A illustrates medical toilet 600 which includes arm rests 140a and 140b. Wrist band 610a is disposed on distal end 150a of arm rest 140a and wrist band 610b is disposed on distal end 150b of arm rest 140b. EKG lead 620a is disposed on wrist band 610a and EKG lead 620b is disposed on wrist band 610b. Toilet 600 also include ankle band 630 on the lower left quadrant of toilet base 130. EKG lead 640 is disposed on ankle band 630. EKG leads 620a, 620b and 640 collect measurements and transmit the measurements to controller 190 for analysis and storage.

FIG. 6B illustrates a close-up view of distal end 150b of arm rest 140b. User's arm 650 is shown resting on arm rest 140b. Wrist band 610b is engaged around user's arm 650 at the wrist. Accordingly, EKG lead 620b is in contact with user's arm 650.

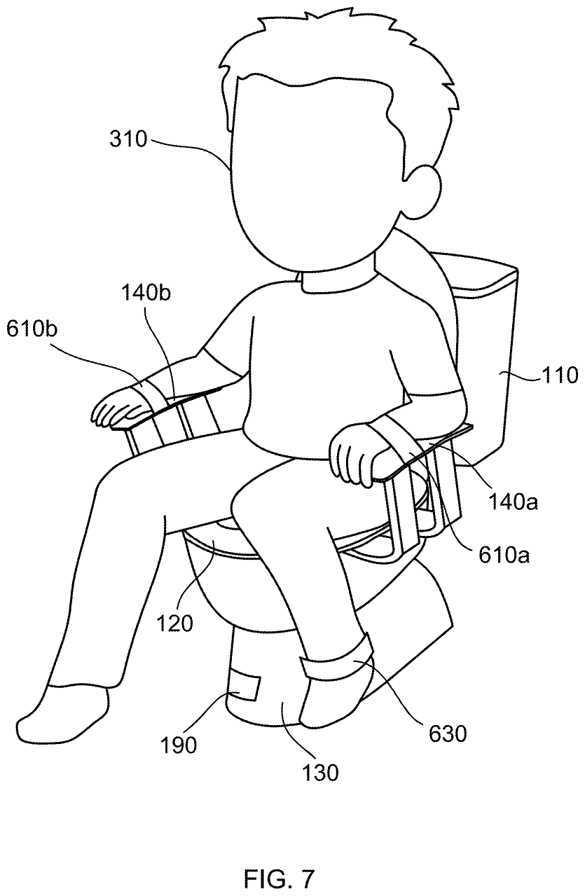

FIG. 7 illustrates medical toilet 600 of FIG. 6A in use by user 310. As user 310 sits on medical toilet 600, wrist bands 610a and 610b are engaged around the wrists of user 310. Accordingly, EKG leads 620a and 620b are in contact with the wrists of user 310. Ankle band 630 is engaged around a left ankle of user 310. Accordingly, EKG lead 640 is in contact with the left ankle of user 310.

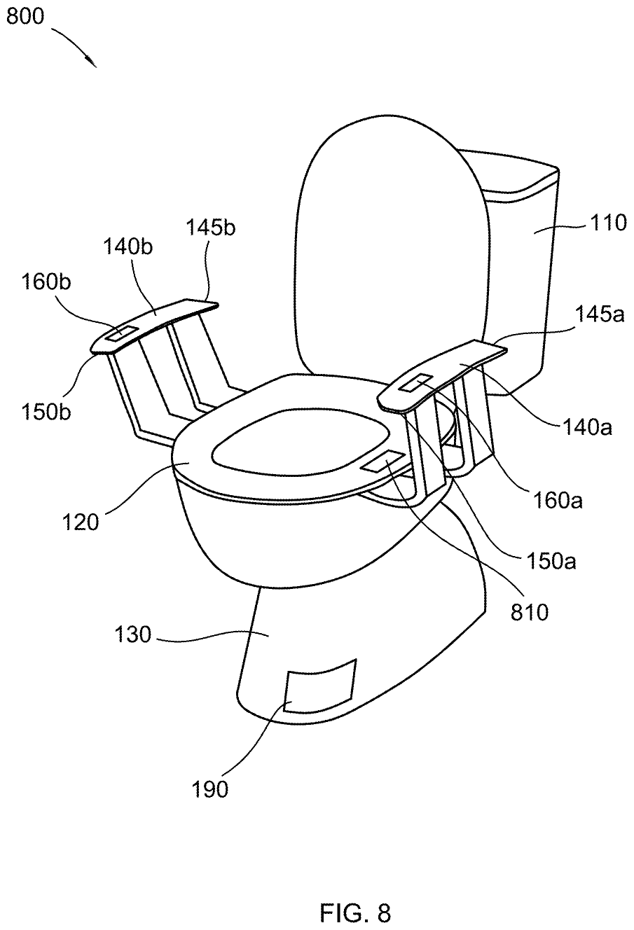

FIG. 8 illustrates medical toilet 800, which includes arm rests 140a and 140b with electrodes 160a and 160b respectively similar to medical toilet 100 of FIG. 1. However, unlike medical toilet 100, medical toilet 800 includes EKG electrode 810 which is disposed on the left side of toilet seat 120. A user may sit on medical toilet 800 and place an arm on each of arm rests 140a and 140b. Each arm will contact EKG leads 160a and 160b. Additionally, the user's left thigh will contact EKG lead 810. EKG leads 160a, 160b and 810 may then collect measurements and transmit the measurements to controller 190 for analysis and storage.

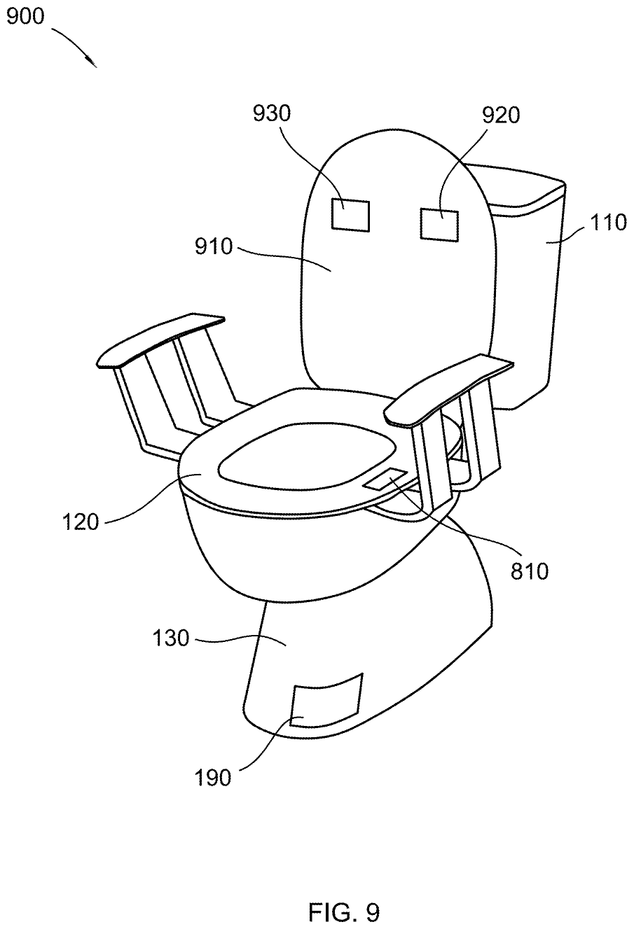

FIG. 9 illustrates medical toilet 900 which includes EKG lead 810 similar to toilet 800. However, medical toilet 900 includes toilet seat 910 which has EKG leads 920 and 930 disposed thereon. A user may sit on medical toilet 900 with his or her left thigh placed on EKG lead 810. The user may lean back against toilet lid placing EKG leads 920 and 930 in contact with the user's back. With the three EKG leads in contact with the user, an EKG reading may be acquired and the measurements transmitted to controller 190 for analysis and storage.

While specific embodiments have been illustrated and described above, it is to be understood that the disclosure provided is not limited to the precise configuration, steps, and components disclosed. Various modifications, changes, and variations apparent to those of skill in the art may be made in the arrangement, operation, and details of the methods and systems disclosed, with the aid of the present disclosure.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art that changes may be made to the details of the above-described embodiments without departing from the underlying principles of the disclosure herein.

We claim:

1. A medical toilet comprising
a. a base;
b. a first and a second arm rest;
c. a controller;
d. a first, a second, and a third electrocardiogram lead, wherein the first, the second, and the third electrocardiogram lead are in electrical communication with the controller; and
e. wherein the first electrocardiogram lead is in connection with the first arm rest and the second electrocardiogram lead is in connection with the second arm rest; and
f. wherein the third electrocardiogram lead is disposed on a left lower quadrant of the medical toilet.

2. The medical toilet of claim 1, wherein the controller comprises a non-transitory computer readable medium, wherein the non-transitory computer readable medium comprises instructions for analyzing a signal collected by the first, the second, and the third electrocardiogram leads.

3. The medical toilet of claim 2, wherein the non-transitory computer readable medium comprises instructions for storing the signal collected by the first, the second, and the third electrocardiogram leads.

4. The medical toilet of claim 2, wherein the instructions comprise steps for determining a user's heart rate.

5. The medical toilet of claim 2, wherein the instructions comprise steps for identifying an irregular cardiac rhythm.

6. The medical toilet of claim 2, further comprising a chest strap, wherein the chest strap comprises a plurality of additional electrocardiogram leads, wherein the plurality of additional electrocardiogram leads comprises precordial leads V1, V2, V3, V4, V5 and V6.

7. The medical toilet of claim 6, wherein the chest strap comprises an elastic fabric.

8. The medical toilet of claim 6, wherein the instructions comprise steps for interpreting the signals from the first, second, and third electrocardiogram leads as either augmented leads aV_{L}, aV_{R}, and aV_{F} or as limb leads I, lead II, and lead III respectively.

9. The medical toilet of claim 8, wherein the non-transitory computer readable medium comprises instructions for interpreting a signal from the first, the second, and the third electrocardiogram leads and the precordial leads V1, V2, V3,V4, V5, and V6.

10. The medical toilet of claim 9 wherein the instructions comprise steps for identifying an irregular cardiac rhythm.

11. The medical toilet of claim 1, further comprising a foot pad, the foot pad comprising the third electrocardiogram lead, wherein the third electrocardiogram lead is disposed on a left side of the footpad, and wherein the foot pad is disposed in front of the toilet bowl and communication with the controller.

12. The medical toilet of claim 1, further comprising a first and a second hand grip, wherein the first hand grip is in mechanical connection with the first arm rest, and wherein the second hand grip is in mechanical connection with the second arm rest, wherein the first electrocardiogram lead is in connection with the first hand grip, and wherein the second electrocardiogram lead is in connection with the second hand grip.

13. The medical toilet of claim 1, wherein the first electrocardiogram lead is disposed on a distal end of the first arm rest, and wherein the second electrocardiogram lead is disposed on a distal end of the second arm rest.

14. The medical toilet of claim 1, further comprising a first and a second finger slip, wherein the first electrocardiogram lead is in connection with the first finger slip, and wherein the second electrocardiogram lead is in connection with the second finger slip.

15. The medical toilet of claim 14, wherein the first finger slip further comprises a pulse oximeter.

16. The medical toilet of claim 1, wherein the third electrocardiogram lead is disposed on a left side of the toilet base.

17. The medical toilet of claim 1 wherein the first, second electrocardiogram leads are each disposed within a wrist band, and wherein the third electrocardiogram lead is disposed within an ankle band.

18. A medical toilet comprising
a. a base;
b. a toilet lid;
c. a toilet seat;
d. a controller; and
e. a first, a second, and a third electrocardiogram lead, wherein the first, the second, and the third electrocardiogram lead are in electrical communication with the controller;
f. wherein the first and the second electrocardiogram leads are disposed on the toilet lid.

19. The medical toilet of claim 18, wherein the third electrocardiogram lead is disposed on the toilet seat.

20. The medical toilet of claim 19, wherein the non-transitory computer readable medium comprises instructions for storing the signal collected by the first, the second, and the third electrocardiogram leads.
